(19) United States
(12) Patent Application Publication
(10) Pub. No.: US 2009/0142384 A1
(43) Pub. Date: Jun. 4, 2009

(54) Viscoelastic material

(76) Inventors: Rolf Muller, Zurich (CH); Federico Innerebner, Zurich (CH)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/292,697

(22) Filed: Nov. 24, 2008

Related U.S. Application Data
(63) Continuation-in-part of application No. 12/230,038, filed on Aug. 21, 2008.
Application No. 12/230,038 is a continuation of application No. 10/515,929, filed on Jun. 8, 2005, now abandoned.

(30) Foreign Application Priority Data
Mar. 28, 2003 (DE) 103 14 418.8
Jun. 18, 2003 (DE) 103 27 870.2

(51) Int. Cl. A61K 8/11; C08B 33/00; A61K 9/48; C08B 31/00

(52) U.S. Cl. 424/401; 536/102; 525/54.24; 524/1; 424/451; 523/100

(57) ABSTRACT

The invention relates to a tough-elastic material based on starch, which on the one hand has high impact toughness at low humidities, and on the other hand still has a high modulus of elasticity at high humidities and has a high elongation capacity in a broad range of humidities and on account of its property profile is suited to use as molded elements such as for example for foils, films, fibers, injection-molded articles, in particular as edible film and for the packaging of active ingredients, chemicals, aromas and perfumes as well as high-quality substitution of gelatine in the area of soft and hard capsules. The tough-elastic material can be obtained transparent and adjusted such that it dissolves on swelling in water or respectively disintegrates or remains intact.

### CROSS-REFERENCE TO RELATED APPLICATIONS

This is a continuation-in-part of application Ser. No. 12/230,038 filed on Aug. 21, 2008 which is a continuation of Ser. No. 10/515,929, a national phase application of International Application No. PCT/CH2004/000191 filed Mar. 26, 2004. International Application No. PCT/CH2004/000191 claims priority to German Application No. 103 12 418.8 filed Mar. 28, 2003 and German Application No. 103 27 870.2 filed Jun. 18, 2003. The contents of the above-noted applications are expressly incorporated herein by reference in their entirety.

### FIELD OF THE INVENTION

The invention relates to a tough-elastic material based on starch, which on the one hand has high impact toughness at low humidity, and on the other hand still has high modulus of elasticity at high humidity and has high elongation capacity in a wide range of humidities.

### PRIOR ART

Different tests were undertaken to obtain a useful material based on starch, based almost exclusively on softened thermoplastic starch (TPS). Polyols are typically used as softeners. In the case of TPS the starch is almost completely in amorphous form. The properties of amorphous polymers are determined predominantly by the brittle temperature Tg. Below Tg the state is vitreous, hard and brittle, and above Tg soft. The difference between both these states is particularly outstanding with TPS. Since starch macromolecules are relatively stiff and rigid, large proportions of softener are required. Below Tg TPS is extremely brittle and in particular very sensitive to a high stress rate, and above Tg TPS more and more takes on the character of a sticky high-viscosity liquid with increasing temperature. Because starch and its softeners are therefore strongly hydrophilic, TPS absorbs water from the atmosphere and the sensitivity of TPS to humidity Relative humidity (RH) is a further problem, which stands in the way of using TPS in practice. The correlation between RH and water content of a material is described by its sorption curve. Through water uptake Tg is thrust down to lower temperatures, so that at a constant temperature with increasing water content a comparable variation of the property profile is obtained, such as with increase in temperature, i.e. at lower RH TPS is hard and brittle, and soft at high RH. As a result of the sorption behavior the material properties such as for example impact toughness K, strength σₘ, modulus of elasticity, elongation capacity ε_{b}, oxygen permeability P_{O2} and surface quality are very noticeably dependent on humidity, whereas ideally the most constant possible material properties are preferred. To date based on starch it has not been possible at low RH to obtain adequate toughness and at the same time to obtain adequate strength at high RH; for this starch had to be blended with synthetic substances. Examples of TPS with the abovementioned disadvantages are specified in patent documents WO 94/28029, U.S. Pat. No. 5,362,777, U.S. Pat. No. 5,382,611, U.S. Pat. No. 5,427,614, WO 94/04600, U.S. Pat. No. 5,415,827 and U.S. Pat. No. 5,462,980.

Soft and hard capsules are a proven form for pharmaceuticals and nutritionals. Once the capsules are ingested the fastest possible release of the capsule contents should generally take place. Accordingly the materials, with which soft and hard capsules are manufactured, or which are potentially considered for this, are at least hydrophilic, generally also water-soluble, such as for example gelatine, which is used to produce more than 95% of the current capsules. The above problem of the material properties varying strongly with RH also applies to these fields of application. Gelatine for example was the previous standard solution in the region of soft and hard capsules, containing 25-50% glycerol as softener, has at RH of 23% 4.5% water, while the water content at RH of around 85% is above 30%. Since water is a very efficient softener, the properties of softened gelatine are thus highly dependent on humidity. Their modulus of elasticity for example, a measurement for stiffness and dimensional stability, is around 85% RH by a factor of around 600 times less than at 23% RH, i.e. at low humidity the material is comparatively stiff and hard, whereas at high RH it becomes very soft and dimensional stability suffers. Further important material properties vary as a function of RH likewise by orders of magnitude. The increase of stickiness and oxygen permeability P_{O2}, which at an increase of RH of 0% to 75% is a factor of approximately 100, is particularly problematic. For these reasons the use of gelatine capsules in particular in damp climates is problematic and expensive packing is required to protect the capsules from moisture.

The pronounced dependence of the properties of hydrophilic capsule materials on humidity is a basic problem. An ideal solution in the area of hard and soft capsules with constant properties in a wide range of current humidities is a priori not possible. In practice there must always be a compromise between the properties at low and at high humidities, i.e. tough behavior at low RH signifies a reduced dimensional stability at high RH and vice versa good dimensional stability at high RH means a loss in toughness to brittle properties at low RH. With gelatine-based capsules at least one acceptable compromise could be found. However since the gelatine obtained from slaughterhouse waste as a result of the BSE problem and in the course of the trend to vegetarian products is being increasingly declined by consumers, the quest was made for new solutions based on raw materials of plant origin. In patent document WO 01/37817 a soft capsule based on thermoplastic starch (TPS) with high softener content is described. It has however the considerable disadvantage of having noticeable brittleness at low humidities, so that in a dry environment the TPS soft capsule already breaks and splinters at minimal stress with a vitreous break. At high RH the TPS soft capsule becomes very soft and sticky and loses its dimensional stability. The TPS soft capsule is therefore clearly the basis of the gelatine soft capsule and the use of the TPS soft capsule is feasible only at average RH. In the case of hard capsules, where the requirements for toughness are even greater as a result of the stress of the capsules in automatic high-speed filling machines, capsules based on TPS could not previously be made. In patent documents U.S. Pat. No. 6,214,376 and U.S. Pat. No. 6,340,473 soft capsules based on carrageenan and starch are described. The disadvantage of this solution is that soft capsules at average RH are already too soft and thus insufficiently dimensionally stable. At higher RH this behavior is even more noticeable. Further disadvantages are the high oxygen permeability, the high raw material costs of carrageenan, clearly more expensive than gelatine, as well as the suspicion of cancerogenity of carrageenan.

These examples clarify the underlying problem of the material properties varying noticeably with humidity in capsules, which apply for other applications of hydrophilic materials in the area of foils, films, fibers, cast articles etc.

Therefore, the object of the present invention is to provide a material having at least the following properties: A transition RH_{Z} of brittle to tough behavior at <33% relative humidity (RH) and room temperature and a modulus of elasticity E of 0.1 MPa<E<50 MPa at 85% relative humidity (RH) and room temperature. A material can be described as tough, if the impact toughness is at least 20 mJ/mm².

### BRIEF DESCRIPTION OF THE INVENTION

This object is archived by a tough-elastic material based on starch with a transition RH_{Z} of brittle to tough behavior and an impact energy in the impact test of K>20 mJ/mm₂ at <33% relative humidity (RH) and room temperature and a modulus of elasticity E of 0.1 MPa<E<50 MPa at 85% relative humidity (RH) and room temperature, characterized in that said tough-elastic material comprises 5-60 weight % dsb of at least one softening agent with a melting point of <70° C. and said tough-elastic material has an amylose content A_{M} of 1-70 weight % dsb wherein said amylose is selected from SCA with a degree of polymerization DPn <100 and a proportion PSCA of SCA specific to amylopectin and SCA of 1-35 weight % dsb, LCA with a degree of polymerization DPn of 100-3'000 and a proportion PLCA of LCA relative to amylopectin and LCA is 1-70 weight % dsb, or a mixture of such SCA and such LCA.

Typically a tough elastic material according to the invention will have dimensional stability at RH in the range of 10-90%, in particular at high RH, toughness at RH in the range of 10-90%, in particular at low RH, long-term stability or respectively resistance to aging, gas barrier properties, in particular low oxygen permeability, good optical properties (transparency and achromatism, but colorable and printable) and it will be biodegradable, in particular edible.

If required, the material according to the invention may be optimized to have the following properties: elasticity of at least 100% in the range of 25-60% RH, weldability, in particular at low temperatures below 40° C., swelling capacity, in particular solubility or respectively disintegration in water, solubility or respectively disintegration in the stomach (37° C.), in particular release of a substance according to pharmacopoeia. The specified properties are not independent, partially even to a large extent mutually dependent, i.e. optimizing a specific property has advantageous or disadvantageous consequences with respect to the other properties. However, such optimization is possible due to the tough-elastic properties of the inventive material over a wide range of RH.

A further advantage of the inventive material is that raw materials available at least in food quality.

With respect to softener (WM) there is a broad palette of known starch softeners to choose from, which have been described numerous times in the prior art (cf. for example WO 03/035026 A2 or WO 03/035044 A2); examples here are the polyols glycerol, erythritol, xylitol, sorbitol, mannitol, galactitol, tagatose, lactitol, maltitol, maltulose, isomalt. These and other softeners can generally be used alone or in diverse mixtures. However, according to the invention it has been found that the desired characteristics of the tough-elastic material could only be obtained by using softeners with melting points <70° C., while use of softeners with melting points >70° C. result in brittle products with low impact energy (K) at 33% RF. Softeners according to the invention are preferably polyols, wherein glycerol (melting point=18° C.) is particularly preferred.

It was further found that according to the invention a softener content of 5-60 weight % dsb is necessary. Preferably the tough-elastic material comprises 10-50 weight % dsb of the softener. A softener content of 15-50 weight % dsb is particularly preferred.

Preferably a tough-elastic material according to the invention comprises an amylose with a degree of polymerization DPn of 10 to 500 wherein the proportion of said amylose relative to total starch is 1 to 15 weight % dsb. Furthermore a tough-elastic material according to the invention preferably comprises an amylose with a degree of polymerization DPn in the range 300 to 2,000 wherein the proportion of said amylose relative to total starch is 1 to 30 weight % dsb, preferably 1 to 27 weight % dsb. Higher amylose contents of said starches typically result in brittle products which also have low solubility.

It is further preferable that a tough-elastic material according to the invention comprises a present starch PS and a network-capable starch NS, wherein the proportion PNS of NS relative to NS and PS is 2-50 weight % dsb and wherein the network-capable starch NS is selected from SCA, LCA or a mixture of SCA and LCA and the present starch PS is selected from the group of oxidated starches, esterificated starches, etherificated starches, hydroxypropylated starches, hydroxyethylated starches, methyl starches, allyl starches, tripheylamethyl starches, carboxymethyl starches and crosslinked starches.

It is also preferable that a tough-elastic material according to the invention has a present starch PS and a network-capable starch NS, wherein the proportion PNS of NS relative to NS and PS is 2-50 in weight % dsb and wherein the network-capable starch NS is selected from SCA, LCA or a mixture of SCA and LCA and the present starch PS has an amylose content of less than 25%.

Details on the function of PS and NS will be given in the detailed description of the invention.

### DETAILED DESCRIPTION OF THE INVENTION

The basis of the tough-elastic material according to the invention is given by a hydrophilic phase, which is water-soluble or swells and decomposes in water. This phase is amorphous or if it is in partial crystalline state, the crystallites or ordered regions are <500 nm. If they have larger dimensions transparency and achromatism cannot be obtained. Amorphous phases generally display brittle behavior at temperatures below the brittle temperature Tg. Since the brittle temperature varies for different properties and the tough-elastic material is used in a limited temperature range at room temperature, instead of the temperature dependency of the brittle-tough transition the dependency of this transition is considered as a function of the RH. At the same time RH_{Z} is the RH whereby at RT the transition from brittle to tough behavior takes place. RH_{Z}<33% therefore applies for the amorphous phase for a material tough at low RH. Thus the amorphous phase with the specified RH exhibits tough behavior. Adjustment of this state is enabled by a selected portion of softener. A polyol or a mixture of polyols with the lowest possible melting points is preferably used as softener, because it has been found that their softening effect is maximal and correspondingly minimal quantities must be employed. A high proportion of softener reinforces the dependency of the properties of the RH.

Amorphous phases behave at temperatures >>Tg or respectively at RH>>RH_{Z} in the manner of highly viscous liquids, also when their viscosity is so high that they appear as solid bodies. Since water is more efficient compared to other softeners in hydrophilic systems with respect to the softening effect by factors, this leads to the fact that the amorphous phase becomes continuously softer with increasing humidity, loses stability and finally deliquesces.

Since an amorphous phase cannot therefore meet the requirements of dimensional stability in the range of 10-90%, toughness in the range of 10-90% and elasticity of at least 100% in the range of 25-60% at high RH, reinforcement was sought. It was found that a starch network can be built for this purpose, which has less dependency of the properties on the RH, since flowing at high RH as a result of cross-linking is not possible. This network interpenetrates the amorphous phase preferably and is linked to this phase. Since existing i.e. chemical networks are water-insoluble from the forming of covalent bonds and also do not disintegrate after swelling, according to the present invention a network is introduced whereof the linking points are thermoreversible and/or can be dissolved again via a solvent, in particular by addition of water or respectively gastric juice at 37° C., or respectively become mechanically unstable. In addition, networks which swell sufficiently are also suitable, so that in the swollen state they disintegrate under the effect of minimal stress. This is possible in particular with thin films. If the network points are formed at least partially by ordered areas such as crystallites, these areas are <500 nm to ensure transparency.

Through water absorption from the atmosphere a network is influenced slightly only with respect to mechanical properties. Whereas for example the modulus of elasticity of a hydrophilic amorphous phase can vary by a factor of around 1000 in the range of the usual humidities, the modulus of elasticity of the network varies by a factor of <10, and in a broad range it can even be virtually constant. The network density is adjusted according to the present invention such that the contribution of the network to the modulus of elasticity and the strength at high water content is at least comparable to the contribution of the amorphous phase. Preferably the contribution of the network in this range is clearly greater than the contribution of the amorphous phase. This even made it possible to obtain virtually constant moduli of elasticity in the range of humidities of approximately 30-70%. The unsatisfactory properties of the amorphous phase at high humidities could be compensated by a network with adequate network density and at the same time toughness could be obtained at low humidities and strength at high humidities.

However, since networks are disadvantageous with respect to water solubility, according to the present invention either the network density is set so low that the network disintegrates after swelling in water as a result of minimal strength under minimal stress (which is the case in particular with thin films), or the network points were preferably adjusted by very small crystallites, which are dissolved in excess by water.

The structure, after having been adjusted, remains stable under alternating conditions of humidity and temperature in an unusually broad range. This can be achieved by formulation and manufacturing conditions, whereby the network density is adjusted to the required volume.

The specified elements basically point out the way to different practicable solutions based on different raw materials and formulations. The salient points are the balance between amorphous phase and network, and the parameter of the network, which on the one hand is sufficiently strong to ensure the mechanical properties of the material under variable conditions and on the other hand does not disable the solubility or disintegration of the capsules in water or respectively in gastric juice. Previous networks corresponding to the prior art based on starch for example are practically completely insoluble in water are stable against disintegration, are known to be opaque to full intransparency, not weldable, also show only minimal elasticities in the region of typically <50% and have an advantageous effect on toughness. An essential key to the solution of the above-mentioned problem is the size of the ordered areas, which constitute the network points. This size can be adjusted by the structure parameter of the raw materials used, in particular by the choice of network-active chain length CLn,na of the used starch molecules.

### Basis and Present Starches

For the base a present starch (PS) is selected. Basically this can be any starch of any origin or a combination of such starches. Yet many starches form no homogeneous amorphous structure. In particular starches containing amylose tend to retrogradation, resulting in an ordered area, often with dimensions >500 nm. On the one hand the transparency is thereby impaired (opacity), and on the other hand retrograde starches exhibit restricted solution or disintegration behavior. Since water solubility can additionally be aggravated by introducing a network, the best possible solution or disintegration behavior of the base or respectively of the amorphous phase is a substantial prerequisite.

Retrogradation is primarily the consequence of the amylose portion of starches, whereby the amylose at least partially crystallizes. For this reason PS or mixtures of PS with an amylose content of <30%, in particular <27% are preferred, i.e. rice or sago starches or starches originating from bulbs and roots such as for example potatoes, yams, canna, arrowroot or tapioca. Likewise waxy starches are preferred, such as for example waxy maize, waxy rice, waxy millet, waxy barley, waxy potato or heterowaxy starches such as for example heterowaxy millet. It has to be noted that lower amylose content will typically result in higher toughness.

With respect to purity starches originating from roots and bulbs or waxy starches are likewise preferred, in particular tapioca starch, since their protein and lipid contents are lower compared to non-waxy wheat starches, which is i.a. also an advantage for transparency and clarity. The disadvantage of wheat starches and potato starches, in particular maize starch, is that different genetically modified variants of these starches are added on and purity with respect to GMO proportions is problematic a priori. Therefore from this viewpoint starches are preferred, whereof no GMO variants are added on, for example sago or root starches, in particular tapioca starches. With respect to technological suitability however genetically modified starches are also considered as PS.

Of particular interest also are dextrins, in particular pyrodextrins such as white dextrins, yellow or respectively canary dextrins, modified dextrins, co-dextrins or British gums. They exhibit good film development properties and as a result of their irregular structure and the high degree of branching Qb of typically >0.05 they are partially to practically fully stable with respect to retrogradation and thus highly water-soluble, as well as being long-term stable, i.e. resistant to aging. Plus, the use of dextrins has a positive effect on the quality of the weld joint of soft capsules, since they have good adhesive properties. Dextrins with low to average degrees of converting can be used as sole PS or can be sued together with other PS, while dextrins with high degrees of conversion are preferably used together with other PS. With regard to optical properties white dextrins are preferred.

Apart from amylose amylopectin can also retrograde, though to a clearly lesser extent and on a clearly larger time scale. The extent of the retrogradation of amylopectin and the stability of retrograded amylopectin regions relative to solubility or respectively disintegration in water is determined by the length of the A side chains of amylopectin. In this context the shortest possible A side chains are advantageous. From this viewpoint starches with weight means of chain length (CLw)<18 are preferred, preferably <16, more preferably <14, in particular <13, most preferably <12, i.e. for example waxy starches, in particular waxy rice, tapioca starches or sago starches. On the other hand the length of the A side chains is also reflected in the more easily measured properties of Blue Value (BV) and Iodine Affinity (IA), so that PS with amylopectin fractions of low BV or respectively low IA are preferred.

Starches or mixtures of such starches, which have been altered and stabilized against retrogradation by subsequent treatment or combinations of treatment, are further preferred as PS, whereby starches with a priori slight inclination to retrogradation such as for example bulbs or roots starches are preferably used: Oxidation (for example periodate oxidation, chromic acid oxidation, permanganate oxidation, nitrogen dioxide oxidation, hypochlorite oxidation: oxidized starches); esterification (for example acetylated starches, phosphorylated starches (monoester), starch sulphate, starch xanthate); etherification (for example hydroxyalkyl starches, in particular hydroxypropyl or hydroxyethyl starches, methyl starches, allyl starches, triphenylmethyl starches, carboxymethyl starches, diethylaminoethyl starches); cross-linking (for example diphosphate starches, diadipate starches); graft reactions; carbamate reactions (starch carbamates).

Starches with partially substituted hydroxyl groups show high elongation for the use of advantageous film formation properties, as required in particular for the production of films and as a result of substitution they are stabilized with respect to retrogradation, i.e. water-soluble and transparent. These properties positive in terms of the invention usually increase with the degree of substitution DS and the size of the substituted group. Starches with DS >0.01, more preferably >0.05, in particular >0.10, most preferably >0.15 are therefore preferred. The upper limit is in each case given by regulatory determinations for food starches. In the technological respect however modified starches with higher DS are also suitable and beneficial.

Examples for substituted starches of particular interest are hydroxypropylated or hydroxyethylated or acetylated or phosphorylated or oxidized roots and bulbs, starches or waxy starches with degree of substitutions of around 0.20 maximal permissible for food starches.

Likewise of particular interest with respect to viscosity are stabilized PS, i.e. chemically cross-linked starches such as for example distarch phosphates, distarch adipates or inhibited starches (Novation Starches). Particularly preferred are chemically cross-linked and at the same time substituted starches, whereby higher degrees of substitution are preferred here also. Appropriate procedures, in particular controlling of shearing forces, can result in at least part of the chemical cross-linking within the starch grain in the end product remaining intact. In this case the amorphous phase is a two-phase system containing network fragments of the original starch grains, by which modulus of elasticity and strength of the capsule can be influenced positively in the problematic area of high humidities, whereas water solubility is not noticeably impaired. Here it should be emphasized that the discontinuous network fragments differ fundamentally from the physical networks essential for the solution. On the basis of network fragments alone the required property profile cannot be achieved, however it can make a positive contribution in terms of an optimised solution. A further advantage of using substituted and at the same time chemically cross-linked starches is that a broad palette of types with different degrees of substitution and cross-linking of these favourable commodity starches are obtainable commercially in foodstuff quality. Examples are hydroxypropylated distarch phosphate, hydroxypropyated distarch adipate, acetylated distarch phosphate or acetylated distarch phosphate, which are obtainable based on starches of different origin such as maize, wheats, millet, rice, potato, tapioca etc.

A further group of interesting starches is hydrolyzed starches such as acid-hydrolyzed starches or enzymatically hydrolyzed starches, as well as chemically modified hydrolyzed starches, in particular based on starches with amylose contents of <25%, as long as they have a reduced inclination to retrogradation, obtained through additional modification such as for example oxidation or substitution.

PS with minimal, reduced or diminishing inclination to retrogradation are primarily preferred. PS with higher amylose contents such as for example wheat starches, pea starches or high-amylose maize starch can however be employed, if measures are taken to prevent or minimise retrogradation such as for example via procedures such as freezing of the amorphous state and/or heat treatment with defined water content, in particular at low water content, and/or chemical modification of PS such as for example substitution of hydroxyl groups, and/or measures concerning formulation, whereby retrogradation-inhibiting materials are added in. Through a combination of these measures on the one hand an amorphous state can be achieved, whereby water solubility and disintegration is ensured, or on the other hand retrogradation can be minimised to the extent that forming a restricted though defined network is still possible, resulting in a balance between toughness at low humidity and adequate strength and stiffness at high humidity. In this case it is possible to dispense with an additional network, which is introduced through the addition of network-capable starch (NS), i.e. the required material properties can then be achieved based solely on PS or a combination of PS. Usually however a combination of PS and NS is used, since the procedural conversion and control of the material properties (solubility, toughness, elongation, transparency etc.) of such mixtures is easier.

The specified present starches can be used both in native granular form (cooking starches), as well as physically modified (pregelatinised, cold-water-soluble, cold-water-swelling).

The methods for selecting the PS, whereby a specific PS or a combination of two or more PS is considered for this purpose, make it clear here that with respect to the origin and type and degree of modification or modifications there is a large number of different possibilities with individual advantages and disadvantages to choose from, whereby technological disadvantages can be compensated by choice of further formulation parameters and/or by procedures. Accordingly it is possible to select as PS a starch or a combination of starches, which satisfy not only the technological requirements, but also commercial aspects such as raw material price and availability, as well as aspects concerning optimal procedural variants, purity or freedom from GMO can be considered. It is also possible to select an optimal solution in each case with respect to the product properties for specific applications.

### Network and Network-Capable Starches (NS)

Since it is not possible on the basis alone of an amorphous phase to obtain adequate toughness at low RH and at the same time to set adequate dimensional stability and strength at high RH, a defined network is introduced, by which the structure is reinforced, preferably creating networks, to which the amorphous phases are linked. This linking can be achieved by a suitable choice of NS and by matching the NS to the PS under suitable procedural conditions.

Starches containing or comprising amyloses or amylose-like starches are employed as NS. A mixture of different NS types is also designated as NS.

Amyloses can be both linear and branched and modified if required. Examples for NS are amyloses from native starches, in particular amyloses obtained through fractionating of starches with an amylose content >23%, modified amyloses, in particular substituted amyloses or hydrolysed amyloses, synthetic amyloses, cereal starches, pea starches, high-amylose starches, in particular with an amylose content >30, preferably >40, more preferably >60, most preferably >90, hydrolysed starches, in particular hydrolysed high-amylose starches or sago starches, gelling dextrins, fluidity starches, microcrystalline starches, starches from the field of fat replacers. Also, NS can also have an intermediate fraction, such as are contained for example in high-amylose starches and can be obtained through fractionating. With respect to its structure and properties the intermediate fraction lies between amylose and amylopectin.

For amylose the distinction in Long Chain amylose (LCA) with DPn >100 and Short Chain amylose (SCA) with DPn <100 is usual. Network-capable starches can have LCA and/or SCA.

### Short Chain Amylose (SCA)

Examples for SCA are amylodextrins, linear dextrins, Nägeli dextrins, lintnerised starches, erythrodextrins or achrodextrins, which represent different descriptions and subgroups of SCA.

SCA can be obtained for example from hydrolysis of LCA, LCA amylopectin mixtures or amylopectin mixtures. For advantageous networks particularly suitable SCA is obtained for example from hydrolysis of starches stemming from roots and bulbs or from heterowaxy or waxy starches. Hydrolysis can take place chemically, such as for example acid hydrolysis, and/or enzymatically such as for example by means of amylases or combinations of amylases (alpha-amylase, beta-amylase, amyloglucosidase, isoamylase or pullulanase). Amylose-containing starches are obtained by combined acid/enzyme hydrolysis as SCA, whereby both hydrolyses can take place at the same time or successively. Depending on this various types of SCA can be obtained starting out from the same starch. In addition the characteristics of SCA are also influenced by the state of the native starch during hydrolysis, for example by the degree of swelling of the starch grains. Therefore there is a broad palette of suitable SCA available. Further types can be obtained by acid/enzyme hydrolysis or enzyme hydrolysis from waxy starches, whereby SCA hydrolysates are obtained with DPn typically around 22, which are particularly suitable. Furthermore, SCA is of particular interest, as it forms during the process of preparation of the starches into NSF and finally into the starch network, for example via pullulanase.

### Long Chain Amylose (LCA)

Amylose contained in native starch is usually LCA with DPn >100. The degree of polymerisation DPn of LCA can however be reduced for example via acid hydrolysis and/or enzymatic hydrolysis and/or oxidation to values <100, so that correspondingly modified native starches can also have SCA.

Countless methods for producing SCA, LCA and mixtures of SCA and LCA are described in the prior art. Both amylose types are obtainable on the one hand in pure form, as well as contained in different, if required hydrolyzed, commercial starches at differing proportions.

### Advantageous Networks

The structural prerequisites for linking the network to the amorphous or respectively predominantly amorphous phase are given by the chain lengths CLw (A-AP) of the A side chains of the amylopectin fraction and by the chain lengths of the amylose fraction. The chain lengths CLw(A-AP) of A side chains of amylopectin for amylopectins from starches with an amylose content <30 lie in the range of around 10-20, whereas high-amylose starches have somewhat higher chain lengths CLw(A-AP). Amyloses by comparison can also have very much higher chain lengths CLw(AM). For Long Chain amyloses (LCA) chain lengths CL(LCA) are typically in the region of 100-1000, whereby roots and bulb starches have clearly higher chain lengths than cereal starches. For Short Chain amyloses (SCA) the chain lengths CL(SCA) are <100 and as a rule are approximately the same size as the degrees of polymerisation DP(SCA), whereby CL(SCA)<DP(SCA). Since only in rare cases are there data on the average weight value CLw for different starches, the numbering means CLn of the chain length distribution or respectively the numbering means DPn of the distribution of the degree of polymerisation is used for simplified discussion. Generally CLw is somewhat greater than CLn, whereby the difference at A side chains of amylopectin is minimal only, since these have a narrow distribution, while the difference at SCA is greater and at LCA can be very great.

The minimal chain length of amylose CLn(AM) or respectively the minimal degree of polymerization of amylose DPn(AM), to obtain linking of a network to the amorphous phase by means of amylose, is approximately CLn(AM)˜CLn(A-AP), i.e. approximately 10-20, whereby advantageous linkings up to approximately CLn(AM)˜100 are possible. Above this value networks can also be created, which are not linked to the amorphous phase, i.e. they predominantly comprise amylose. With respect to the set requirements these networks have disadvantageous properties, for example opacity at higher RH, water insolubility, compared to linked networks of clearly reduced elongation at breaks and toughnesses.

For this reason SCA is suited as NS or as a portion of NS for the production of networks linked to the amorphous phase, whereby the stability of the crystallites forming the network points, i.e. their size, decreases with decreasing CLn(AM) or respectively DPn(AM) and the water solubility and transparency of the substance increases.

Advantageous networks are obtained with proportions PSCA of SCA in % by weight dsb relative to amylopectin and SCA is in the region of 1-35, preferably 2-25, in particular 3-20, most preferably 4-14.

Furthermore, advantageous linking of the network to the amorphous phase using LCA is also possible, whenever its network-active chain length CLn,na(LCA) is in the range of the chain length of SCA, i.e. <100.

In the chain length CLn(AM) irregularities can be introduced by chemical reactions, in particular by substitution of hydroxyl groups of the anhydroclucose monomer unit, by oxidation or cross-linking. In a chemical reaction at the centre of mass of a segment characterized by its chain length CL the network-active chain length of CL is halved to ½CL. Therefore it is possible to obtain advantageous networks, for example via hydroxypropylising or acetylising, also based on LCA. Advantageous degrees of substitution (DS) are in the region of approximately 0.01-0.50.

Advantageous networks are obtained with proportions PLCA of modified LCA in % by weight dsb relative to amylopectin and LCA in the region of 1-70, preferably 2-50, in particular 3-40 more preferably 4-35, most preferably 5-30. At high degrees of modification the proportions PLCA are at higher values as compared to lower degrees of modification.

Finally, advantageous networks based on LCA with CLn,na >100 can be obtained, if suitable conditions for this are created by procedures, such as for example forming at comparatively low water contents or respectively low temperatures and/or heat treatment at RH in the range of 20-60% and/or addition of retrogradation-inhibiting materials (RIM), whereby the (large-space) association of amylose with amylose networks is suppressed and the (small-space) association of amylose with A side chains of amylopectin is favoured.

Preferably the inventive tough-elastic material has a starch with a network-active chain length CLn,na, whereof the length is in the range of 5-300, preferably 6-100, more preferably 7-50, in particular 8-30, most preferably 9-28, most particularly von 10-27, whereby the material if required has a strongly branched other starch with a degree of branching Qb >0.01, preferably >0.05, more preferably >0.10, most preferably >0.15.

Preferably the inventive tough-elastic material has a PS and a NS, whereby the proportion PNS of NS relative to NS and PS in % by weight dsb is in the range of 1<PNS<90, preferably 2<PNS<50, more preferably 3<PNS<30, most preferably 3<PNS<15.

The inventive tough-elastic material is characterized advantageously by the property that the material:
a) has an amylose content A_{M} in % by weight dsb in the region of 2<A_{M}<50, preferably 3<A_{M}<40, more preferably 3<A_{M}<30 and
b) the amylose is SCA, LCA or a mixture of SCA and LCA, whereby the proportion PSCA of SCA in % by weight dsb relative to amylopectin and SCA is in the region of 2-25, in particular 3-20, most preferably 4-14; and/or the proportion PLCA of LCA in % by weight dsb relative to amylopectin and LCA is in the region of 2-50, in particular 3-40 more preferably 4-35, most preferably 5-30.

The inventive tough-elastic material is further characterized advantageously by the property that:
a) the SCA has a degree of polymerisation DPn in the range of 5<DPn<70, preferably 6<DPn<50, in particular 7<DPn<30, more preferably 8<DPn<28, most preferably 9<DPn<27; and
b) the LCA has a degree of polymerisation DPn in the range of 100<DPn<3,000, preferably 100<DPn<1000, more preferably 100<DPn<500, most preferably 100<DPn<300; and
c) if required the LCA has a degree of substitution DS in the range of 0.01-0.50, preferably 0.02-0.30, more preferably 0.03-0.25, most preferably 0.04-0.20.

To set a defined network NS is activated with PS prior to or during mixing and in particular stabilized. The activating ensures that the amylose contained in NS is in the amorphous state, so that recombination can take place after the molecular dispersing mixture with PS to a network-capable starch fluid (NSF), which leads to a network in which both NS and PS participate. At the same time the network development is induced by the crystallization capacity of NS raised following activation. The stabilizing enables influencing of the beginning of network development and the type of network.

The higher the water content and the greater the shearing forces during plasticizing or dissolving procedure, the lower the necessary temperatures. Of particular significance is activation connected to stabilisation of NS. Stabilisation is achieved by overheating of the amylose to temperatures above the melting or dissolving procedure. Through stabilization the temperature of the recombination of amylose can be adjusted to the desired network at low temperatures. The higher the stabilizing or respectively the overheating temperature, at a lower temperature with the same water and softener content the recombination or respectively the network development takes place. Furthermore, foreign nucleating means and/or methods can be employed for producing suitable nuclei by means of undercooling the activated NS. With respect to activation stabilizing, formation of nuclei, undercooling and foreign nucleating means reference is made to patent applications WO 03/035026 A2 and WO 03/035044 A2 for detailed data, where the preparation of NS prior to mixing with PS, the mixing procedure and the continuously following forming and network development are described (Split Continuous Process, SCP).

A further advantageous method is that production of a preproduct takes place after the mixing procedure, for example in the form of granulate or powder. This preproduct can later be prepared again and processed into an end product (Split Discontinuous Process, SDP). The production of different preproducts and other methods of operating, where NS and PS can also be prepared together (Together Continuous Process, TCP and Together Discontinuous Process, TDP) are described in patent application WO 2004/085482 A2 establishing priority for the present application with publication date of Jul. 10, 2004 and included per reference in this patent application.

### Solubility and Disintegration in Aqueous Media

Through introducing a network it is possible to adjust high softener contents, by which the brittleness of the capsules can be overcome at low humidities and at the same time the mechanical properties in the range of high humidities are still guaranteed. Since known networks however impair transparency and lead to water insolubility, two basic requirements are thus not fulfilled.

This problem was able to be solved on the one hand in that networks with lower network densities are set, whereby transparency is barely impaired, the network can disintegrate in the swollen state in water and another adequate contribution to the mechanical properties is guaranteed in particular at high humidities. The clearance is however restricted and the potential of the network cannot be fully utilised. Therefore on the other hand possibilities were sought out to obtain water solubility and transparency at higher network densities also.

As already mentioned, a key role is played by controlling the dimensions of the crystallites constituting the network points. Influence is possible via procedures, in particular via heat treatments and/or via substantial requisites. At average RH and lower temperatures as a result of the restricted diffusion of the macromolecules smaller crystallites are obtained as at higher RH and higher temperatures.

Since SCA with DPn of for example 24 in crystallized form, whereby the SCA is present as helix with around 6-8 monomer units per elongation and a length per elongation of around 0.8 nm, has a length of around 3×0.8 nm=2.4 nm, the minimal size of the combination of such SCA is given with A side chains of crystallites formed by amylopectin with around 2.4 nm, whereby the A side chains are comparable to the SCA. This size is far below that required for transparency 500 nm and such crystallites are also unstable in water excess at 37° C.

With the choice of the molecular weight of SCA both the transparency as well as the water solubility or respectively disintegration in water can therefore be favorably influenced. With increasing DPn of SCA the tendency to form crystallite agglomerates rises, whereby transparency, water solubility and also toughness are impaired. This trend also continues for DPn >100, i.e. for LCA, which is why SCA in particular with lower degrees of polymerization DPn is preferred or higher-molecular amylose with network-active chain length CLn,na correspondingly restricted for example by substitution.

The relationship between the length of linear polymers in the crystalline state and the size of the corresponding crystallites (lamella density) is known in the area of synthetic polymers, however in the range of polysaccharides it has not yet been recognized that this legality can be utilized advantageously, in particular for networks of high mechanical stability and elasticity, which can nevertheless disintegrate in water.

Larger crystallites can arise through agglomerates or through of SCA or LCA with higher DPn. In particular with LCA excessively low degrees of branching Qb can be disadvantageous, lead to opacity and water insolubility, or respectively prevent disintegration after swelling. Transparency and water solubility can also be obtained with higher-molecular SCA and LCA, if for example these amyloses are substituted, the network-active chain length CLn,na is reduced are and/or suitable procedures are undertaken, in particular regulating the water contents down to low values and/or heat treatment at comparatively low RH following production. That means that the same factors, which enable advantageous networks to be set up, in particular networks linked to the amorphous phase, also have a positive effect on water solubility and transparency. Total water solubility is not a required condition for the release of an active ingredient, and disintegration of the material can likewise enable release. In the context of this invention water solubility is also understood to be disintegration, since certain types of the tough-elastic material do not fully dissolve but disintegrate.

Water solubility is determined primarily by the above measures concerning formulation and methods, and secondly a positive influence on water solubility is also possible by using the following materials:

### Retrogradation-Inhibiting Materials (RIM)

RIM can be used advantageously both for tough-elastic materials based on PS alone or a combination of PS and NS. At the same time materials basically come from good water solubility, which are miscible with a network-capable starch fluid (NSF). The retrogradation-inhibiting effect of these materials is based on the one hand on reduction of the waters available for the starch as softener, and in the diluting of the starch phase, whereby diffusion of the starch macromolecules is made difficult in both cases, and the existing incompatibility of RIM and starch with respect to crystallisation. Examples for suitable RIM are types of sugar such as glucose, galactose, fructose, sucrose, maltose, trehalose, lactose, lactulose, raffiniose, glucose syrup, high maltose corn syrup, high fructose corn syrup, hydrogenised starch hydrolysate and also polydextrose, glycogen, oligosaccharides, mixtures of oligosaccharides, in particular with DE >20, preferably >25, more preferably >30, most preferably >70, maltodextrins, dextrins, pyrodextrins, in particular with degrees of branching Qb >0.05, preferably >0.10, more preferably >0.15, most preferably >0.3.

RIM additionally improve per se the water solubility, partially influence the sorption behavior favorably and in particular the types of sugar considerably lower the oxygen permeability, which is why they are also particularly advantageous for this reason. If retrogradation-inhibiting materials are incapable of fully suppressing retrogradation, dextrins, pyrodextrins, maltodextrins, oligosaccharides and glycogen in particular enable control of the dimensions of the crystallites resulting from retrogradation to dimensions where transparency is not impaired and water solubility or respectively disintegration in water can be accomplished. In those applications where this property is of significance, a proportion PRIM of retrogradation-inhibiting substances (RIM) relative to PS and NS and RIM in % by weight dsb in the region of 1-70, preferably 3-50, more preferably 5-25, most preferably 7-20 is effectively used.

### Explosives (E)

Explosive or disintegration accessories used in galenic according to the prior art are considered as explosives, in particular fillers, which develop a gas on absorption in water and/or swell strongly, by means of which the network mechanically destabilises and disintegrates. Examples are carbonates and hydrogen carbonates of alkali and earth alkali ions, in particular calcium carbonate, as well as soya proteins (for example Emcosoy) or preferably strongly swelling starch particles such as sodium glycolates (sodium salt of carboxy methyl ether starch), for example Explotab, Vivastar or Primojel. Furthermore, salts also come into consideration.

A proportion PE of explosive (E) relative to PS and NS and E in % by weight dsb in the region of 0.1-30, preferably 0.5-15, more preferably 1-10, most preferably 1.5-7.0 is added if required to improve the disintegration performance.

### Solvents (S)

Solvents are understood in particular as non-starch polysaccharides or respectively hydrocolloids, which have good water solubility or strong swelling capacity in water and are miscible with NS and/or PS or are present therein as separate phase. If necessary, a proportion pₛ of solvent (S) relative to PS and NS and S in % by weight dsb in the region of 1-50, preferably 2-25, more preferably 3-20, most preferably 4-15 is added to improve water solubility or swelling capacity.

### Optical Properties

Measures enabling solubility of networks in water or respectively gastric juice at 37° C. also enable adjusting the transparency, which is problematic with standard networks (opacity). The corresponding measures have already been mentioned. This can be obtained up to ca. 85% and high transparency of high quality, comparable to gelatine. Whereas gelatine has a yellowish to brownish innate color, films comprising the tough-elastic material are practically entirely colorless. If pyrodextrins with yellowish to brownish coloring are used in clear proportions, the result is approximately the coloring of gelatine.

Common natural or synthetic dyes can be used for coloring, as used for example for colouring gelatine capsules.

As for printing capacity starch offers advantages compared to gelatine. This is understandable, since starch is utilized in large quantities in the paper industry, thus improving i.a. the print capacity of paper.

### Surface Properties

Tackiness is reduced prior to beginning the network development compared to gelatine, since at this point gelatine has a very much higher water content. As the network builds the stickiness is continuously reduced, and on completion of the network development there is practically no stickiness.

### Impact Toughness

The same sample can appear tough at a lesser stress rate and at a high stress rate seem brittle. This is particularly the case with substances based on starch and in the area of transition from brittle to tough behavior. Since high stress rates also do occur in practice, impact toughness is decisive. Apart from impact toughness, expressed as energy (impact capacity) relative to the sample cross-section absorbed at break, elongation of the sample to the break ε_{K} is also relevant as a measure for the derformability or respectively toughness in the event of sudden stress. At around 33% RH surprisingly high impact toughnesses K up to 1000 mJ/mm² and more were received by the inventive tough-elastic material based on starch; at elongations ε_{K} of around 25%, during the same conditions TPS has impact toughnesses of typically around 10 mJ/mm² at ε_{K}˜0% and soft capsule gelatines have impact toughnesses around 400 mJ/mm² and ε_{K}˜25%. As already mentioned, the minimal toughness or respectively the distinct brittleness of TPS soft capsules is the central problem, by which the corresponding technology can be utilized, though strongly restricted.

The toughness of TPS and from the inventive tough-elastic material is determined at a specific RH primarily by the brittle temperature Tg. The brittle temperature is a possibility for characterizing a continuous phase transition in amorphous material, characterized by an increase of degrees of freedom of the components resulting for example in heightened thermal capacity, thermal expansion, flexibility or increased toughness, whereby the respective transition temperatures can have clear differences and at a constant temperature a corresponding transition of the property depending on the softener contents can be observed. With respect to toughness depending on the RH at RT, RH_{Z}, transition is decisive for selecting the optimal softeners or the optimal softener combination. For tough starch mixtures RH_{Z} is at <30%, preferably <20%, i.e. at these relatively low RH the material already shows around half the maximal toughness. If glycerol is employed as softener, in the range of 20-40% glycerol, depending on PS, NS and other formulation parameters such as for example retrogradation-inhibiting substances, RH_{Z} is in the region of 15-30%, whereby adequate toughness in the problematic area of the lower RH is guaranteed.

The toughness of the tough-elastic material can also be further improved, in particular at RH<33%, in that a proportion of polyvinyl alcohol (PVA) is added, in particular a proportion in % by weight in the region of 1-50, preferably 1.5-30, more preferably 2-20, in particular 3-15, most preferably 3-10. Basically any PVA types can be considered here, but PVA types with degrees of hydrolysis <90% are preferred, more preferably <80%, whereby PVA preferred is mixed in the NSF in dissolved form.

### Heat Treatment and Resistance to Aging

A method is designated as heat treatment, whereby the material is stored in an atmosphere and the atmosphere has a course of humidity and temperature as a function of time. Using heat treatment the network development and if required the retrogradation can be controlled in the finished capsule. At RT and in the region of approximately 0-30% RH the network development is suppressed, while it runs in the region of approximately 60-90% RH with increasing speed. At too high RH cloudiness can appear which is why heat treatments are carried out advantageously in the average range of humidity. By adjusting temperatures above RT the heat treatment can be shortened, whereby the suitable RH decreases with increasing temperature. The duration of the heat treatment depends on the exact formulation and in particular the degree of polymerization of the amylose and is in the region of hours to days. Here too SCA enables advantages as compared to LCA, i.e. brief heat treatment times. As a result of the greater mobility of the shorter molecules heat treatment can also be omitted.

In addition, heat treatment is carried out to precisely anticipate transposition procedures, which would otherwise run uncontrolled. Constant product properties and long-term stability can be obtained hereby.

### Additives

Additives and/or fillers and/or resistant starches can be added to the tough-elastic material as additives. In this respect reference is made to the patent applications WO 03/035026 A2 and WO 03/035044 A2, as well as to the DE patent application of 28 Mar. 2003 with file number 103 14 418.8 establishing priority for the present application.

The operating costs in the area of soft capsules are comparable up to and including the drying process to the operating costs of gelatine capsules. Since capsules based on the tough-elastic material as compared to gelatine are produced with clearly lower water content the drying process can be reduced. With optimized operating parameters it can even be omitted entirely.

### Raw Materials

The structure selected as a solution to the above task basically allows different conversion possibilities, whereby the parameters of the solution can in each case be adapted and optimized. There is plenty of leeway available for production based on starch with the broad spectrum of commercially available starches (large starch producers typically offer >100 different starches; in total there are >1000 individual starch types and qualities, often with graduated properties, available on the market). Therefore a considerable number of individual solutions is possible by way of specific formulations and adapted operating variants. Different starches for consideration are detailed in the description. In particular also solutions based on favourable quantities of starches (commodity starches) of food quality can be converted and other requirements concerning availability, purity or GMO freedom can be considered in addition to the raw material price, and minor conditions, which can also alter over time. In all the price advantage for solutions based on raw materials of food quality compared to gelatine is significant with a factor of 2-7.

### Applications

On account of the new combinations of properties the inventive tough-elastic material is suited for high-quality soft capsules, which can be used similarly to conventional gelatine soft capsules. The soft capsules can be produced using a continuous encapsulating method such as for example with the rotary die method, whereby the capsule is formed similarly to gelatine encapsulating from films supplied symmetrically to the encapsulating plant, and these films are formed using current standard methods such as for example extrusions or casting methods. Welding is performed at temperatures in ° C. in the range of 10-120, preferably 15-90, more preferably 20-70, most preferably 25-50. Encapsulation takes place directly from the freshly produced films or the films are prefabricated and stored as rolls, before encapsulating. Such a method is very advantageous, though not possible with gelatine capsules. With the production of prefabricated films, even though they are already made with a low water content, heat treatment or conditioning of the soft capsules can be reduced or omitted entirely in contrast to gelatine capsules.

Likewise, the tough-elastic material can be used for high-quality hard capsules, which can be used similarly to conventional gelatine soft capsules. The forming can take place as for gelatine hard capsules in the dip process. In addition forming can be carried out advantageously also via the injection-moulding method, whereby heat treatment or conditioning of the soft capsules can be reduced or omitted entirely in contrast to gelatine capsules.

The tough-elastic material can be in the form of diverse molded articles, in particular foil; film, preferably edible film; filament; fiber, preferably oriented fibers manufactured in the gel spin method; foam; granulate; powder; microparticles; injection-molded item; extruded item; profile-cast article; deep-drawn item; thermoform article.

The uses are many and apply in particular to the foodstuffs, galenic, cosmetic, health care, packaging or agrarian sectors, for example as cotton wool rods, polystyrene foam replacement, foil, bioriented foil, compound foil components, membrane system for nano-, micro- or macroencapsulation, paper laminate, replacement of cellulose, throw-away clothing, crockery and cutlery, food tray, drinking straw, mug, food packing, foamed heat-insulated food container, chew bones for dogs, shopping bag, waste and compost sack, mulch foil, plant pot, golf tip, toy.

### ADVANTAGES OF THE INVENTION

An essential aspect of the present invention is, that a present starch PS by is cross-linked means of a network-capable starch NS to characteristic networks and the brittle temperature Tg of the matrix is lowered by adjusting the softener and the softener contents to the extent where adequate toughness is already obtained at low relative humidities RH and on the other hand as a result of the network also at high RH still adequate strength and elasticity is obtained. This property combination essential for most applications could not previously be achieved with known thermoplastic starch (TPS), which is practically fully amorphous. While the mechanical properties of TPS vary dramatically within the area of usual humidity, even a tough-elastic material with a quasiplateau of mechanical properties, i.e. with useful properties in a broad range of relative humidity RH, was obtained.

The tough-elastic material at low RH has astonishing toughness, which is improved by a factor of >100 for example compared to TPS, where the toughness is critical, i.e. the limiting factor, and at the same time at high RH good dimensional stability, i.e. a high modulus of elasticity can be obtained. With respect to the balance between toughness and dimensional stability even a property profile improved on as compared to gelatine was able to be obtained. Furthermore, lower oxygen permeabilities can be set, by which the spectrum of application possibilities relative to current gelatine and TPS can additionally be improved on (e.g. oxidation-sensitive active ingredients). As a result of the improved sorption behavior the water absorption is also reduced, likewise improving the application possibilities.

In addition the networks can be optimized to specific requirements with respect to their type and shaping. Further modification possibilities will emerge through specific additives. Therefore for example networks can be obtained which become very weak in water and disintegrate or dissolve. The result of this for example is the application of gelatine in soft and hard capsules as replacement. On account of the composition the new material is also eminently suited for edible films. As a result of the network the material is also not tacky at high humidities. This behavior seems minor, but for many applications it is just as essential as the new mechanical property combinations. Likewise, transparency is of major significance for many applications.

The improved sorption behaviour and the reduced oxygen permeability improve for example the service life of capsule formulations (galenics, aroma, perfume). Furthermore, the used starches are widely available and of high purity, as compared to gelatine by a factor of 2 to 7, and finally also the operating costs can be lowered relative to gelatine capsules as a result of a simplified or fully superfluous conditioning procedure and by means of novel methods (production of films for the encapsulating independently of the encapsulating method, preparation of films in the form of rolls). Since different formulations basically enable useful solutions, whereby if required each operating parameter has to be adapted, there is plenty of leeway for individual solutions and aspects such as the raw material price, availability, purity or freedom from GMO, therefore minor conditions, which may alter over time, can also be considered.

Further advantages, features and application options of the invention will emerge from the non-limiting examples and figures.

### FIGURES

FIG. 1: modulus of elasticity as a function of relative humidity. The moduli of elasticity of different modifications of the inventive tough-elastic material are stabilized to high RH at a high level, whereas at low temperatures tough thermoplastic starch (TPS) becomes fluent there and loses mechanical properties. The formulations are listed in Tables 2-5.

FIG. 2: elongation at break as a function of relative humidity.

FIG. 3: modulus of elasticity as a function of relative humidity. Thermoplastic starch can be adjusted to adequate properties either at low or at high RH, while the new tough-elastic material has good properties in the whole area.

FIG. 4: tensile strength at 10% elongation as a function of humidity. The same situation occurs as in FIG. 3.

FIG. 5: impact toughness as a function of relative humidity. Soft thermoplastic starch has high toughness at low RH, but at high RH neither toughness nor modulus of elasticity or strength (FIGS. 3, 4). At high RH the toughness of brittle TPS is adequate, at low RH however minimal. The new material on the other hand shows good properties in both areas.

FIG. 6: modulus of elasticity as a function of relative humidity. Property spectrum of different tough-elastic modifications.

FIG. 7: modulus of elasticity as a function of relative humidity. Compared to the batch method (tough-elastic 1) extrusion provides clearly improved properties with minimal anisotropy of extruded films.

FIG. 8: elongation at break as a function of relative humidity.

FIG. 9: tension as a function of elongation during the tensile test. In the tough-elastic material there is a pronounced elastic limit, a qualitative similarity with polyethylenes for example

FIG. 10: sorption behavior. The sorption behavior is clearly improved relative to gelatine.

FIG. 11: oxygen permeability. The barrier effect is clearly improved relative to gelatine.

### EXAMPLES

### Materials Used

G-Dexis a yellow dextrin based on tapioca starch.

Hydr.1 is a hydrolyzed starch based on potato (E number 1404).

Hydr.2 is a hydrolyzed starch based on potato (E number 1451).

Hydr.3 is a hydrolyzed starch based on potato (E number 1420).

LCA1 is a starch with a DPn of about 180.

LCA2 is a starch with a DPn of about 370.

SCA1 is a starch with a DPn of about 25.

LCA1, LCA2 and SCA1 are laboratory samples made according to M. J. Gidley, Macromolecules 1989, 22, 341-346. Similar starches, which can also be used for the invention, can be obtained by a technique described in U.S. Pat. No. 5,468,286 (but resulting starches don't necessarily need to be obtained in crystalline form) or by other techniques using debranching enzymes and/or alpha amylase well known in the art.

### Batch Method

The batch method was performed by means of a heatable Brabender kneader with a chamber volume of 50 cm³. In a first step a PS according to tables 2-5 was plasticized by addition of water and a softener according to tables 2-5 at mass temperatures of 80-90° C. and 120 rpm for 3 min. Parallel to this a solution a NS according to tables 2-5 was prepared according to table 1 and added to the melt. Homogenizing was carried out at 100 rpm for 10 min, whereby the mass temperature rose continuously to 90-105° C. The finished mixture was then removed and shaped in a press into films of 0.5 mm, which contained typically around 20% water. The films were then stored at various RH to equilibrium and analyzed with respect to K at 33% RF, E at 75% RF and E at 85% RF. Different formulations for tough-elastic materials according to the invention (examples 1-21) are listed in Tables 2-4 and reference materials (comparative examples 1-9) are listed in Tables 5.

It has been found that only the formulations according to the invention showed tough-elastic properties. As can be seen from comparative examples 1-4 use of a PS and glycerol as the softener without adding a NS results in soft products with E=0 at 85% RF (comparative examples 1, 3 and 4) or in brittle products with low K at 33% RF (comparative example 2). On the other hand use of a PS and a NS in combination with a softener with a melting point of >70° C., such as maltitol (melting point 149-152° C.) or sorbitol (melting point 99-101° C.) always resulted in brittle products with low K at 33% RF (comparative examples 5-9).

TABLE 1

| | TL1 | dT/dt | TL2 | C |
|---|---|---|---|---|
| NS type | [° C.] | [° C./min] | [° C.] | [weight %] |
| G-Dex | fehlt | fehlt | fehlt | fehlt |
| Hydr.1 | 185 | 50 | 80 | 14 |
| Hydr.2 | fehlt | fehlt | fehlt | fehlt |
| Hydr.3 | fehlt | fehlt | fehlt | fehlt |
| LCA1 | 190 | 70 | 85 | 12 |
| LCA2 | 195 | 90 | 90 | 10 |
| SCA1 | 175 | 25 | 50 | 30 |

TL1: Solution temperature,
dT/dt: cooling rate of solution,
TL2: temperature of solution on addition to PS melt,
C: concentration of solution

### Continuous Method, Direct Extrusion

Alternatively tough-elastic materials according to the invention may be produced via direct extrusion using the following extrusion parameters: 30 mm twin-shaft extruder turning in same direction, tightly meshing (20L/D), screw configuration: inlet zone, distributive mixture (G3), dispersive mixture (G4), outlet zone (G5), speed 300 rpm, PS=7.1 kg/h (dose G1), NS solution=3.3 kg/h (25% NS, 75% water, dT/dt=50° C./min, dose G2), softener=3.5 kg/h (dose G3), temperature housing G1=40° C., G2=80° C., G3=90° C., G4=90° C., G5=90° C. The final water content after extrusion could be varied by means of a vacuum in the range of 10-30%.

Example 1 has been repeated using said direct extrusion method. The mixture was formed by means of a wide-slot nozzle into a film of 0.6 mm in thickness and calibrated by means of a Chill Roll. The resulting material has the following properties (example 1E): K 33% RF=1120 mJ/mm², E 75% RF=32 MPa and E 85% RF=8 MPa. The foil can then be rolled up and stored, processed further at a later time, or it can also be processed directly for example via a rotary die plant into soft capsules or via a welding and cutting plant into sachets. If the foil is interim stored then the water content should be below around 15% at a softener content of around 25-35% at room temperature, thus the network development does not set in. In terms of water contents of approximately 7-15% there is a very interesting state (presuming there is still no or only a minimally developed network). With these ratios the NSF on the one hand is in a state above the brittle temperature Tg, i.e. the material is relatively soft and shows a very high elongation capacity of typically 300% and more, on the other hand the NS in the NSF remains surprisingly in the molecular dispersed distributed state at least for months, so that the good formability and weldability remain intact for just as long. Following processing the network development can then be triggered by an increase in temperature and/or of water content, whereby the material consolidates as a result of the incipient network development and loses its weldability at low temperatures. It is not yet understood why network development cannot take place under the abovementioned conditions; it is obviously inhibited (in the presence of nuclei however network development is also possible under these conditions), although the material is soft and is above Tg, yet the observed state is technologically of major use, for example with respect to storage capacity and further processing of the material. That the NSF consolidates with an increase in temperature and/or water contents is truly surprising, since the very opposite would be expected, as is also the case with TPS, but it is understandable, since the resulting network has additional strength and this at first glance paradoxical phenomenon thus clearly demonstrates a multiple useful difference between TPS and NSF or respectively the starch network resulting from the NSF.

In an alternative NS solution may be dosed in G3 and softener in G2. In a further alternative the prepared NS solution may mixed with softener (dT/dt=30° C./min) and dosed in G2. In just another alternative NS solution and softener are in each case dosed in G2.

### Properties

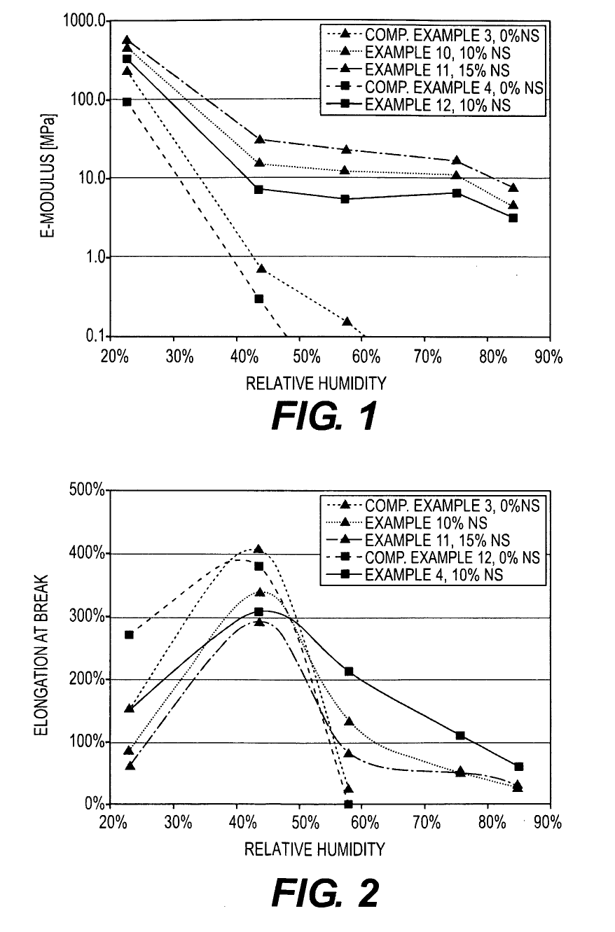

FIG. 1 shows the sequence of the modulus of elasticity as a function of relative humidity for formulations based on retrogradation-stabilised starches (average to high DS), which are particularly suitable for the inventive tough-elastic material as matrix or respectively amorphous phase are and have a an extraordinarily good film-forming capacity. The formulations according to comparative examples 2 and 3 show the basic problem of obtaining a useful material based on starch in a broad humidity range. These materials are relatively impact resistant at low RH of 20-30%, yet water is quickly absorbed with increasing humidity, whereby they already become very soft from ca. 40% RH, lose their solid character and gradually take on the properties of slowly flowing highly viscous liquids. The drop in moduli of elasticity with RH is dramatic. For example the material according to comparative example 4 varies in the RH range 20-40% by virtually a factor of 1000. For each use, subjected to the atmosphere, such materials are conceivably unsuitable.

The formulations according to examples 10, 11 and 12 show a defined network, whereby on the one hand the impact-resistant behavior is not impaired at low RH, but on the other hand the mechanical properties such as for example the modulus of elasticity at average to high RH can be stabilized. Surprisingly even a quasiplateau of the modulus of elasticity was obtained in the RH range of around 40-75%, whereby the modulus of elasticity remains virtually constant. The level of the quasiplateau depends on the one hand on the selected PS and on the type and proportion of the NS. Comparison of the material according to example 10 with 10% NS with the material according to example 11 with 15 NS shows the influence of the NS portion.

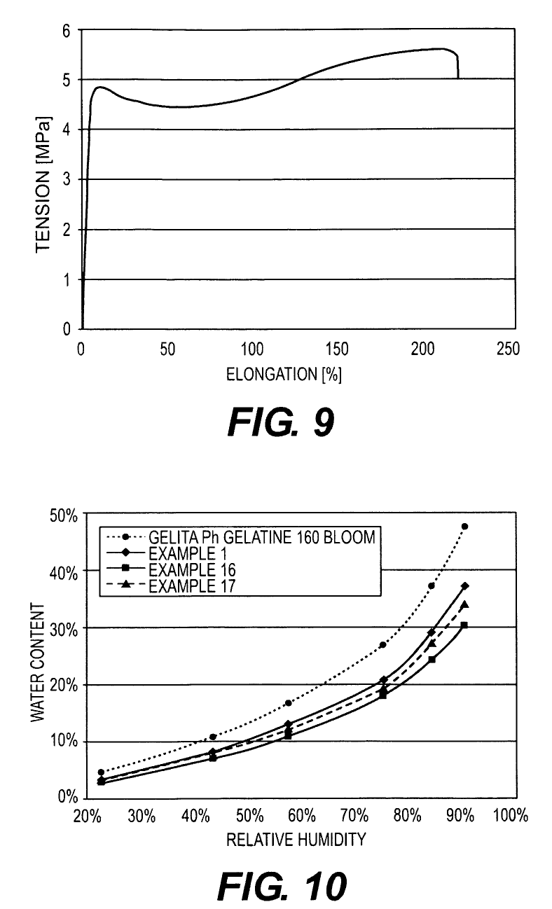

Interestingly, the tension elongation curves of the tough-elastic materials according to the invention show a course in the RH range of around 20-50%, comparable for example with the tension elongation curve of polyethylene, whereby an elastic limit, a subsequent plateau region and finally a consolidation area can be established. In FIG. 9 the tension elongation curve is illustrated for example for a material according to example 10 at RH=33%.

FIG. 2 shows the elongations at break of the formulations of FIG. 1. The elongations at break of the formulations according to examples 10, 11 and 12 show at around 45% RH a maximum of 300% and within a wide range of humidity of approximately 20-70% elongations at break of at least 100% are obtained. This behavior reflects the excellent filming property in a wide water content range. Through use of NS the maximums of the elongation at break relative to formulations without NS are somewhat lower than those of the materials according to comparative examples 3 and 4, however it also shows up here that the range of use to high RH can be expanded partially clearly by introducing a defined network.

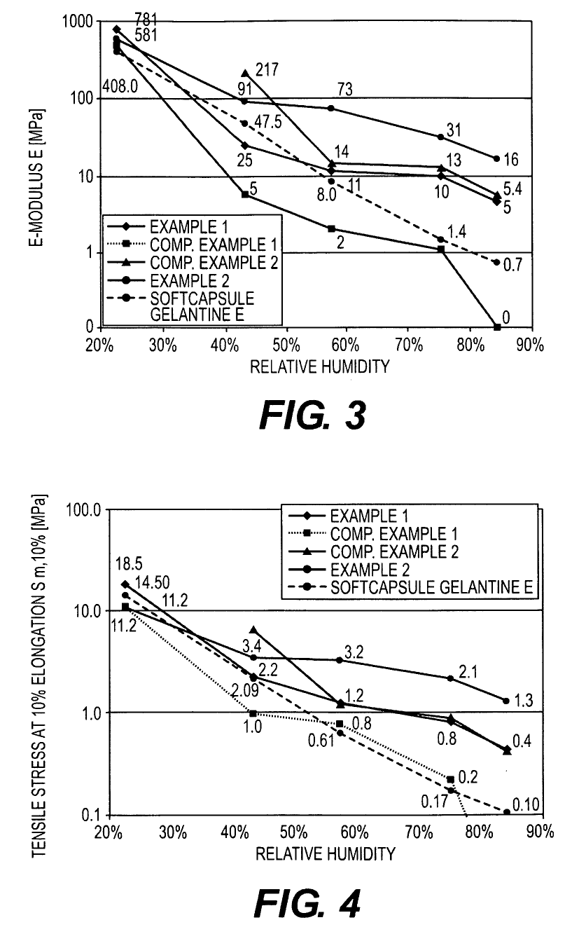

In FIG. 3 the behavior of the moduli of elasticity is shown as a function of the RH for two typical inventive tough-elastic materials (examples 1 and 2) as well as for a soft (comparative example 1) and a brittle TPS (comparative example 2) and for soft capsule gelatine. Soft capsule gelatine in the logarithmic figure shows a linear drop in the modulus of elasticity with increasing RH and at the same time varies in the range of RH of around 20-85% by a factor of around 600. Materials according to examples 1 and 2 in this RH range show a clearly reduced variation width by a factor 100 and in particular a quasiplateau in the average RH range. This is a significant advantage relative to gelatine. Whereas gelatine and tough-elastic materials according to examples 1 and 2 at 22% RH have comparable moduli of elasticity, the moduli of elasticity of materials according to example 1 and 2 at 85% RH are around 10 to 20 times higher, whereby the dimensional stability is clearly improved at high RH.

The material according to comparative example 1 is based on a substituted starch with low DS. This formulation shows what can be achieved in the most optimal case with respect to the modulus of elasticity at high RH, if impact toughness can be obtained at the same time at low RH. The moduli of elasticity at higher RH are modest however, and only a value of 2 MPa is already obtained at 58% RH, while gelatine has 8 MPa and the tough-elastic materials according to examples 1 and 2 still have 11 or respectively 73 MPa. As a result of the low DS the starch used for comparative example 1 is little suited as PS for inventive tough-elastic materials; in particular there has not been sufficient of this property for those applications where disintegration in water is essential. In contrast to comparative example 1 comparative example 2 shows at higher humidities moduli of elasticity, which are comparable to example 1. Yet the impact toughness at 32% RH is extremely low with only 11 mJ/mm² compared to 904 mJ/mm² in example 1, i.e. the material according to comparative example 2 is outstandingly brittle at low RH, and the material breaks like glass at the slightest stress.

The sequence of tensile strength at 10% elongation as a function of the RH for the abovementioned formulation is illustrated in FIG. 4. The ratios with respect to this property are similar to the modulus of elasticity.

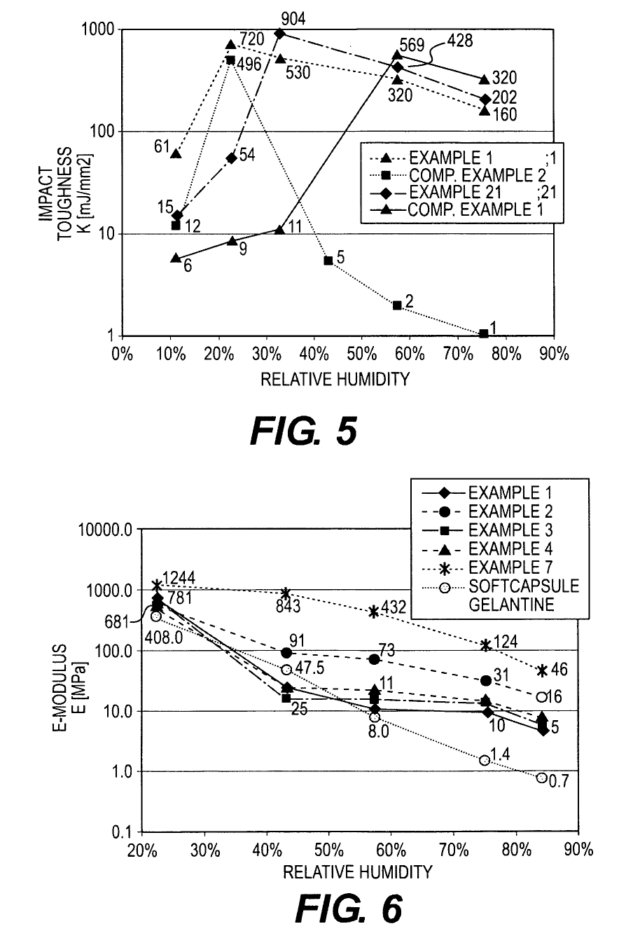

The sequence of the impact toughness or respectively impact energy K as a function of the RH is specified for comparative examples 1 and 2, and for examples 1 and 21 in FIG. 5. A material based on starch can be described as tough, if the impact toughness is at least 20 mJ/mm², yet higher values are an advantage. The material according to comparative example 2 becomes somewhat tough just above 40% RH, whereas the tough-elastic material according to example 1 becomes tough above 20% RH and the material according to example 21 even below 10% RH, therefore is still tough also at extremely low humidity, as normally hardly ever occurs. The transition from brittle to tough takes place in comparative example 1 between 10-20% RH. The following sharp drop in impact toughness at higher RH is based on that fact that the material becomes markedly soft with increasing RH and takes on the character of a highly viscous liquid. In addition to the impact toughness the elongation at break in the impact test ε_{K} is a further measure for characterizing the breaking performance. Whereas the material according to comparative example 2 has no measurable elongation at break, elongations at break of 25% and more could be obtained with tough-elastic material according to the invention, i.e. this material still behaves plastic also at high stress rates.

FIGS. 3, 4 and 5 clearly express a basic problem of TPS. So on the one hand it is possible to set adequate impact toughness at low RH, whereby at high RH the material becomes very soft and fluid (minimal modulus of elasticity), or based on TPS at high RH an adequate modulus of elasticity can be set, whereby the material becomes extremely brittle at low RH. This behavior is based on the fact that TPS is practically fully amorphous, is vitreous below the brittle temperature Tg, and above Tg is present as highly viscous liquid. Useful properties can thus be obtained only in the transition region between both states, within a narrow RH range. In contrast to this with the inventive tough-elastic material both toughness and strength properties (modulus of elasticity, strength, dimensional stability) can be achieved at the same time in a broad RH range, whereby in addition still other properties, as required for specific applications, can be adjusted (e.g. transparency, disintegration in aqueous media, water solubility). It is also of particular advantage that the properties can virtually be stabilized in a RH range of typically 40-75% (quasiplateau of the modulus of elasticity and strength).

FIG. 6 shows the moduli of elasticity for different tough-elastic formulations according to the invention as a function of RH. On the one hand this demonstrates that the characteristic properties of the inventive tough-elastic material can be obtained by means of different formulations, and on the other hand the level of the modulus of elasticity can be varied in a range comprising virtually two decades.

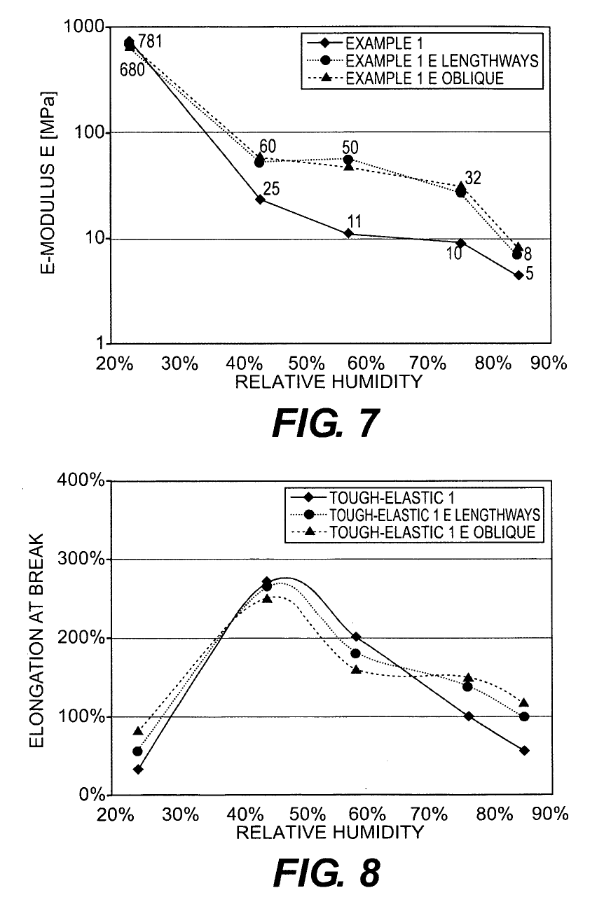

The property profile of the inventive tough-elastic material is not only dependent on the formulation, but also on the production method. Comparison of the properties as produced for the same formulation by means of a batch method (Brabender kneader, example 1) and by means of a continuous extrusion method (example 1E) is evident from FIG. 7. It becomes clear that the modulus of elasticity according to extrusion method in the range of the quasiplateau and above is on a clearly higher level, whereby as compared to example 1 around 3 to 5 times higher values were obtained, i.e. the advantages of the tough-elastic material are even more clearly pronounced with production by extrusion then the results based on the batch method. FIG. 8 shows that the elongation capacity of the tough-elastic material from example 1E compared to the material from example 1 in the range of the maximum at average RH decreases slightly, however increases at low and high RH. The properties resulting from the extrusion method better as compared to the Brabender method are generally usual and based on factors such as for example higher homogenity, fewer material errors, shorter operating times.

FIG. 10 compares the sorption isotherms of tough-elastic according to examples 1, 16 and 17 to the sorption isotherms of gelatine. Gelatine absorbs more water with overall RH range as compared to the tough-elastic material at identical RH. This is one of the reasons why diverse properties of gelatine exhibit higher dependency on the RH. The water absorption of the tough-elastic material can be reduced by specific formulation measures, in particular through the composition of the softener (examples 16 and 17), where different other properties are less dependent on the RH.

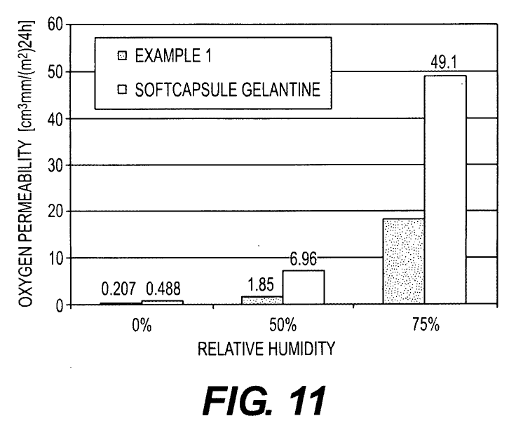

In particular for applications in the encapsulating area, but also in general in the range of packaging, good barrier properties are advantageous compared to gases, in particular compared to oxygen (damage to the contents by oxidation). FIG. 11 shows that the oxygen permeability of tough-elastic material according to example 1 compared to soft capsule gelatine is reduced by a factor of 2 to 3, where a further advantage compared to gelatine is apparent. Oxygen permeability can be further reduced by formulation measures, in particular through the use of sugar. Compared to example 1 example 17 shows oxygen permeabilities in the RH range 0-75% reduced by a factor ½, whereas this factor is even ¼ with example 16.

Based on a typical tough-elastic formulation, modified with the addition of 10% sugar, a film of 0.25 mm thickness was made using a Brabender kneader, producing bags by means of a pulse welding plant, containing fluid aroma concentrates and perfumes. Even after a one-month storage period the bags were still intact and an excellent barrier effect of the tough-elastic material could be ascertained. After the bags were placed in cold water, after 15 min complete disintegration of the bags could be observed, effectively releasing the contents. The result of this for example is the possibility of producing sachets containing perfumes, which to date have comprised polyvinylalcohol and are used in washing machines to obtain washed clothes with a pleasant aroma. The advantage of such bags based on starch is on the one hand price and on the other hand very good biological degradability of starch. In terms of aroma, aroma concentrates can be encapsulated by the tough-elastic material, whereby the release of the aroma occurs on application and up to this time the quality of the aromas can be kept very well protected over a longer time (Top Notes). As compared to previous encapsulating systems in the aroma area here also the stability of the tough-elastic material at high humidities and the absence of stickiness over the entire RH area is a major advantage. Furthermore, the release of medicinal active ingredients from capsules comprising the tough-elastic material was examined, whereby the results corresponded to the requirements according to pharmacopoeia.

### Measuring Methods and Conditioning

### Tensile Test

The tensile tests were determined at 22° C. with an Instron 4502 tensile test machine at a traverse speed of 50 mm/min on standardised tensile samples according to DIN 53504 S3, which were stamped from films of around 0.5 mm thickness. The measuring results are to be understood as average values of in each case at least 5 separate measurements. The water contents of the tensile samples conditioned at different humidities were constant during the duration of the tensile tests within the measuring precision. The tension σ0 was obtained as F/A, whereby F was the force and A the sample cross-section at ε=0. The elongation in the tensile test in % was obtained as ε=100(I₁−I₀)/I₀, whereby Iₒ was the expandable length of the sample between the clamps at the beginning of the tensile tests and I₁ was the length of the expanded sample. The modulus of elasticity was obtained as E=σ|ε.

### Impact Toughness

The impact toughness was determined according to the Izod Impact Method with a Frank Impact Tester (type 53565, Karl Frank GmbH, Weinheim, Birkenau, Germany) with striking pendulums of 4 joules (high impact toughnesses) or 1 joule (low impact toughnesses). As test specimens film samples with 5 mm width and ca. 0.5 mm thickness were used. The length of the samples between clamping on both sides was 40 mm. The elongation at break ε_{K} in the impact test was obtained as ε_{K}=100(I₁−I₀)/I₀, whereby I₀ was the expandable length of the sample between the clamps prior to impact and I₁ was the length of the expanded sample after break. The measuring results are to be understood in each case as average values of at least 5 individual measurements. During the tests the water content of the samples remained constant within the measuring precision.

### Oxygen Permeability

The measurements for oxygen permeability were made with a OX-TRAN 2/21 (MOCON Inc. 7500 Boone Avenue North, Minneapolis, USA) on films of 0.15 mm thick, whereby the oxygen permeabilities of in each case starch film and gelatine film were measured in a symmetrical arrangement at the same time, so that the relative values could be determined very precisely.

### Sorption

The sorption measurements were taken on samples (square sample bodies of 5 mm edge length and 0.5 mm thick) previously dried to 0% water content (24 h at 75° C. on phosphorpentoxide), which were then stored at different RH, which were adjusted by saturated salt solutions, for 7 days in desiccators. The desiccators were fitted with ventilators, by which the sorption times could clearly be shortened to equilibrium (7 days) as compared to storage in still atmosphere. The water contents after sorption were determined by the loss of water during subsequent drying.

### Conditioning

The conditioning of the samples for mechanical analyses (tensile test, impact toughness) was performed in the same equipment as used for sorption (7 days).

TABLE 2

| Example | | | | | | | | |
|---|---|---|---|---|---|---|---|---|
| 1 | 2 | 3 | 4 | 5 | 6 | 7 | 8 | 9 |

Present starch (PS)

| | | | | | | | | | | |
|---|---|---|---|---|---|---|---|---|---|---|
| Pea | [weight %] | | | | | | | 60 | | |
| Potato (HA) | [weight %] | | | | | | | | | |
| Potato (HP) | [weight %] | | | | | | | | | |
| Tapioca (ADSP) | [weight %] | 58 | | | | | | | | |
| Tapioca (HP) | [weight %] | | | 53 | | | | | | |
| Tapioca (HPDSP) | [weight %] | | | | | 58 | 58 | | | |
| Waxy Maize | [weight %] | | 53 | | | | | | | |
| Waxy Maize (ADSA) | [weight %] | | | | | | | | | |
| Waxy Maize (ADSP) | [weight %] | | | | | | | | | 58 |
| Waxy Maize (HPDSP) | [weight %] | | | | 53 | | | | | |
| Waxy Rice | [weight %] | | | | | | | | 58 | |
| Waxy Rice (ADSP) | [weight %] | | | | | | | | | |

Network-capable starch (NS)

| | | | | | | | | | | |
|---|---|---|---|---|---|---|---|---|---|---|
| G-Dex | [weight %] | | | 15 | | | | | | |
| Hydr.1 | [weight %] | | | | | | | | | |
| Hydr.2 | [weight %] | | | | | | | | | |
| Hydr.3 | [weight %] | | | | | | | | | |
| LCA1 | [weight %] | | | | | 10 | | | 10 | 10 |
| LCA2 | [weight %] | | | | | | 10 | | | |
| SCA1 | [weight %] | 10 | 15 | | 15 | | | 15 | | |

Softening agent (SA)

| | | | | | | | | | | |
|---|---|---|---|---|---|---|---|---|---|---|
| Glycerol | [weight %] | 32 | 32 | 32 | 32 | 32 | 32 | 25 | 32 | 32 |
| Maltitol | [weight %] | | | | | | | | | |
| Sorbitol | [weight %] | | | | | | | | | |
| Sugar 1 | [weight %] | | | | | | | | | |
| Sugar 2 | [weight %] | | | | | | | | | |

Impact energy and E-modulus

| | | | | | | | | | | |
|---|---|---|---|---|---|---|---|---|---|---|
| K 33% RF | [mJ/mm²] | 904 | 237 | 178 | 144 | 900 | 650 | 62 | 650 | 232 |
| E 75% RF | [MPa] | 10 | 31 | 13 | 14 | 5 | 5 | 124 | 22 | 12 |
| E 85% RF | [MPa] | 5 | 16 | 5.4 | 8 | 3 | 3 | 46 | 11 | 7 |

Modification:
HPDSP = hydroxypropyated distarch phosphate,
HP = hydroxypropyated starch
ADSP = acetylated distarch phosphate,
ADSA = acetylated distarch adipate,
HA = hydrolysed acetylated

TABLE 3

| Example | | | | | | | | |
|---|---|---|---|---|---|---|---|---|
| 10 | 11 | 12 | 13 | 14 | 15 | 16 | 17 | 18 |

Present starch (PS)

| | | | | | | | | | | |
|---|---|---|---|---|---|---|---|---|---|---|
| Pea | [weight %] | | | | | | | | | |
| Potato (HA) | [weight %] | | | | | 58 | | | | |
| Potato (HP) | [weight %] | | | | | | | | | |
| Tapioca (ADSP) | [weight %] | | | | 58 | | | | | |
| Tapioca (HP) | [weight %] | | | | | | | | | |
| Tapioca (HPDSP) | [weight %] | | | 58 | | | | | 55 | 58 |
| Waxy Maize | [weight %] | | | | | | | | | |
| Waxy Maize (ADSA) | [weight %] | | | | | | | | | |
| Waxy Maize (ADSP) | [weight %] | | | | | | | | | |
| Waxy Maize (HPDSP) | [weight %] | 58 | 53 | | | | 55 | 58 | | |
| Waxy Rice | [weight %] | | | | | | | | | |
| Waxy Rice (ADSP) | [weight %] | | | | | | | | | |

Network-capable starch (NS)

| | | | | | | | | | |
|---|---|---|---|---|---|---|---|---|---|
| G-Dex | [weight %] | | | | | | | | |
| Hydr.1 | [weight %] | | | | | | 10 | 10 | |
| Hydr.2 | [weight %] | | | | | | | | 10 |
| Hydr.3 | [weight %] | 10 | 15 | | | | | | |
| LCA1 | [weight %] | | | | | | | | |
| LCA2 | [weight %] | | | | | | | | |
| SCA1 | [weight %] | | | 10 | 10 | 10 | | | |

Softening agent (SA)

| | | | | | | | | | | |
|---|---|---|---|---|---|---|---|---|---|---|
| Glycerol | [weight %] | 32 | 32 | 32 | 32 | 32 | 25 | 25 | 25 | 25 |
| Maltitol | [weight %] | | | | | | | | | |
| Sorbitol | [weight %] | | | | | | | | | |
| Sugar 1 | [weight %] | | | | | | 10 | 7 | | |
| Sugar 2 | [weight %] | | | | | | | | 10 | 7 |

Impact energy and E-modulus

| | | | | | | | | | |
|---|---|---|---|---|---|---|---|---|---|
| K 33% RF | [mJ/mm²] | 367 | 144 | 763 | 939 | 376 | 217 | 168 | 142 |
| E 75% RF | [MPa] | 10 | 16 | 6 | 11 | 49 | 11 | 17 | 10 |
| E 85% RF | [MPa] | 4.4 | 8 | 3 | 7 | 26 | 6 | 12 | 5 |

Modification:
HPDSP = hydroxypropyated distarch phosphate,
HP = hydroxypropyated starch
ADSP = acetylated distarch phosphate,
ADSA = acetylated distarch adipate,
HA = hydrolysed acetylated

TABLE 4

| Example | | |
|---|---|---|
| 19 | 20 | 21 |

Present starch (PS)

| | | | | |
|---|---|---|---|---|
| Pea | [weight %] | | | |
| Potato (HA) | [weight %] | | | |
| Potato (HP) | [weight %] | | | |
| Tapioca (ADSP) | [weight %] | | | |
| Tapioca (HP) | [weight %] | | | |
| Tapioca (HPDSP) | [weight %] | 63 | | 54 |
| Waxy Maize | [weight %] | | | |
| Waxy Maize (ADSA) | [weight %] | | 58 | |
| Waxy Maize (ADSP) | [weight %] | | | |
| Waxy Maize (HPDSP) | [weight %] | | | |
| Waxy Rice | [weight %] | | | |
| Waxy Rice (ADSP) | [weight %] | | | |

Network-capable starch (NS)

| | | | | |
|---|---|---|---|---|
| G-Dex | [weight %] | 5 | | 12 |
| Hydr.1 | [weight %] | | | |
| Hydr.2 | [weight %] | | | |
| Hydr.3 | [weight %] | | | |
| LCA1 | [weight %] | | | |
| LCA2 | [weight %] | | | |
| SCA1 | [weight %] | | 10 | |

Softening agent (SA)

| | | | | |
|---|---|---|---|---|
| Glycerol | [weight | 32 | 32 | 34 |
| Maltitol | [weight %] | | | |
| Sorbitol | [weight %] | | | |
| Sugar 1 | [weight %] | | | |
| Sugar 2 | [weight %] | | | |

Impact energy and E-modulus

| | | | | |
|---|---|---|---|---|
| K 33% RF | [mJ/mm²] | 772 | 205 | 530 |
| E 75% RF | [MPa] | 4 | 13 | 9 |
| E 85% RF | [MPa] | 1 | 6 | 5 |

Modification:
HPDSP = hydro xypropyated distarch phosphate,
HP = hydro xypropyated starch
ADSP = acetylated distarch phosphate,
ADSA = acetylated distarch adipate,
HA = hydrolysed acetylated

TABLE 5

| Comparative Example | | | | | | | | |
|---|---|---|---|---|---|---|---|---|
| 1 | 2 | 3 | 4 | 5 | 6 | 7 | 8 | 9 |

Present starch (PS)

| | | | | | | | | | | |
|---|---|---|---|---|---|---|---|---|---|---|
| Pea | [weight %] | | | | | | | | | |
| Potato (HA) | [weight %] | | | | | | | | | |
| Potato (HP) | [weight %] | | 63 | | | 58 | 50 | 43 | | |
| Tapioca (ADSP) | [weight %] | 68 | | | | | | | | |
| Tapioca (HP) | [weight %] | | | | | | | | | |
| Tapioca (HPDSP) | [weight %] | | | | 68 | | | | 53 | 53 |
| Waxy Maize | [weight %] | | | | | | | | | |
| Waxy Maize (ADSA) | [weight %] | | | | | | | | | |
| Waxy Maize (ADSP) | [weight %] | | | | | | | | | |
| Waxy Maize (HPDSP) | [weight %] | | | 68 | | | | | | |
| Waxy Rice | [weight %] | | | | | | | | | |
| Waxy Rice (ADSP) | [weight %] | | | | | | | | | |

Network-capable starch (NS)

| | | | | | | |
|---|---|---|---|---|---|---|
| G-Dex | [weight %] | | | | | |
| Hydr.1 | [weight %] | | | | | |
| Hydr.2 | [weight %] | | | | | |
| Hydr.3 | [weight %] | | | | | |
| LCA1 | [weight %] | | | | | |
| LCA2 | [weight %] | | | | | |
| SCA1 | [weight %] | 10 | 10 | 10 | 10 | 10 |

Softening agent (SA)

| | | | | | | | | | | |
|---|---|---|---|---|---|---|---|---|---|---|
| Glycerol | [weight %] | 32 | 37 | 32 | 32 | | | | | |
| Maltitol | [weight %] | | | | | | | 24 | | 37 |
| Sorbitol | [weight %] | | | | | 32 | 40 | 23 | 37 | |
| Sugar 1 | [weight %] | | | | | | | | | |
| Sugar 2 | [weight %] | | | | | | | | | |

Impact energy and E-modulus

| | | | | | | | | | | |
|---|---|---|---|---|---|---|---|---|---|---|
| K 33% RF | [mJ/mm²] | 841 | 11 | 454 | 458 | 5 | 8 | 6 | 7 | 5 |
| E 75% RF | [MPa] | 1 | 13 | 0 | 0 | 11 | 13 | 11 | 10 | 12 |
| E 85% RF | [MPa] | 0 | 5.4 | 0 | 0 | 3 | 5 | 4 | 3.5 | 4.3 |

Modification:
HPDSP = hydroxypropyated distarch phosphate,
HP = hydroxypropyated starch
ADSP = acetylated distarch phosphate,
ADSA = acetylated distarch adipate,
HA hydrolysed acetylated

Symbols and Abbreviations

| | | |
|---|---|---|
| RH | [%] | relative humidity: 0% < RH < 100% |
| RT | [° C.] | room temperature (22° C.) |
| Tg | [° C.] | brittle temperature |
| WM | [%] | softener content (excluding water) relative to starch and softener, dsb |
| W | [%] | water content, relative to starch, softener and water |
| dsb | [—] | dry solid base, relative to the dry weight |
| E | [MPa] | modulus of elasticity (Young's Modulus) |
| σₘ | [MPa] | maximal strength in the tensile test (break resistance) |
| σ_{10%} | [MPa] | tensile strength in the tensile test at ε = 10% |
| ε_{b} | [%] | elongation at break in tensile test |
| F_{E(23-85)} | [—] | variation width of the modulus of elasticity in the range of RH of |
| | | 23-85%, F_{E(23-85)} = E₂₃/E₈₅. |
| F_{E(43-75)} | [—] | variation width of the modulus of elasticity in the range of RH of |
| | | 43-75%, F_{E(43-75)} = E₄₃/E₇₅. |
| F_{σ10%(23-85)} | [—] | variation width of σ_{10%} in the range of RH of 23-85%, Fσ_{10%(23-85)} = |
| | | σ_{10%,23}/σ_{10%,85} |
| F_{σ10%(43-75)} | [—] | variation width von σ_{10%} in the range of RH of 43-75%, Fσ_{10%(23-85)} = |
| | | σ_{10%,23}/σ_{10%,85} |
| K | [mJ/mm²] | impact energy in the impact test (Izod Impact Test) |
| ε_{K} | [%] | elongation at break in the impact test (Izod Impact Test) |
| RH_{Z} | [%] | RH at transition from brittle to tough behavior at RT. K(RH_{Z}) |
| | | becomes the arithmetic means of toughness of the plateau in the |
| | | brittle area K_{S and maximal toughness K}_{M} defined according |
| | | to the brittle-tough transition. Since as a rule K_{S} << K_{M} is K(RH_{Z}) ~½K_{M} |
| P_{O2} | | [ml × cm/(cm² × 24 h × atm)] permeability coefficient for oxygen |
| A_{M} | [% by weight] | amylose content, relative to starch, dsb |
| p_{NS} | [% by weight] | proportion of NS relative to NS and PS, dsb |
| p_{LCA} | [% by weight] | proportion of LCA in % by weight dsb relative to AP and LCA |
| p_{SCA} | [% by weight] | proportion of SCA in % by weight dsb relative to AP and SCA |
| p_{RIM} | [% by weight] | proportion of RIM, relative to PS and NS and RIM |
| p_{E} | [% by weight] | proportion of E, relative to PS and NS and E |
| p_{S} | [% by weight] | proportion of S, relative to PS and NS and S |
| DP | [—] | degree of polymerization |
| DPn | [—] | numbering means of the degree of polymerization |
| DPw | [—] | weight means of the degree of polymerization |
| Qb | [—] | degree of branching of macromolecules (number of branched |
| | | monomer units/number of monomer units) |
| CL | [—] | chain length (number of units) |
| CLn | [—] | numbering means of the chain length; linear, i.e. unbranched chain segments |
| CLn, na | [—] | numbering means of network-active chain length; chain segments, |
| | | which crystallize and can participate in networks, i.e. unbranched and |
| | | non-substituted and non-sterile impeded chain segments |
| CLw | [—] | weight means of chain length |
| DS | [—] | degree of substitution: 0 < DS < 3.0 |
| DE | [—] | dextrose equivalent: 0 < DE < 100 |
| BV | [—] | Blue Value |
| IA | [g/100 g] | iodine affinity |
| PS | | present starch |
| NS | | network-capable starch |
| WM | | softener, can be individual softener or a mixture of different softeners |
| RIM | | retrogradation-inhibiting materials (RIM) |
| E | | explosive |
| S | | solvent |
| AM | | amylose |
| AP | | amylopectin |
| A-AP | | A side chains of amylopectin |
| SCA | | Short Chain amylose (NS or proportion of NS) with DPn in the |
| | | region of 10-100; SCA can alone form no starch networks, only in |
| | | combination with other starches of a higher degree of polymerization, |
| | | networks comprising such mixtures can still be formed at lower |
| | | softener content and low temperatures |
| LCA | | Long Chain amylose (NS or proportion of NS) with DPn > 100 |
| NSF | | network-capable starch fluid; melt or solution containing a starch |
| | | or a starch mixture and softener; can then be obtained as starch |
| | | network under appropriate conditions. |

1. A tough-elastic material based on starch with a transition RH_{Z} of brittle to tough behavior and an impact energy in the impact test of K>20 mJ/mm² at <33% relative humidity (RH) and room temperature and a modulus of elasticity E of 0.1 MPa<E<50 MPa at 85% relative humidity (RH) and room temperature, characterized in that
a) said tough-elastic material comprises 5-60 weight % dsb of at least one softening agent with a melting point of <70° C. and
b) said tough-elastic material has an amylose content A_{M} of 1-70 weight % dsb wherein said amylose is selected from SCA with a degree of polymerization DPn <100 and a proportion p_{SCA} of SCA specific to amylopectin and SCA of 1-35 weight % dsb, LCA with a degree of polymerization DPn of 100-3'000 and a proportion p_{LCA} of LCA relative to amylopectin and LCA is 1-70 weight % dsb, or a mixture of such SCA and such LCA.

2. A tough-elastic material based on starch as claimed in claim 1, characterized in that it comprises 10-50 weight % dsb of the softening agent (SA).

3. A tough-elastic material based on starch as claimed in claim 2, characterized in that it comprises 15-50 weight % dsb of the softening agent (SA).

4. A tough-elastic material based on starch as claimed in claim 1, characterized in that the softening agent (SA) with a melting point of <70° C. is a polyol.

5. A tough-elastic material based on starch as claimed in claim 4, characterized in that the softening agent (SA) with a melting point of <70° C. is glycerol.

6. A tough-elastic material based on starch as claimed in claim 1, characterized in that said material comprises an amylose with a degree of polymerization DPn of 10 to 500 wherein the proportion of said amylose relative to total starch is 1 to 15 weight % dsb.

7. A tough-elastic material based on starch as claimed in claim 1, characterized in that said material comprises an amylose with a degree of polymerization DPn in the range 300 to 2,000 wherein the proportion of said amylose relative to total starch is 1-30 weight % dsb.

8. A tough-elastic material based on starch as claimed in claim 7, characterized in that the proportion of said amylose with a degree of polymerization DPn of 300 to 2,000 relative to total starch is 1-27 weight % dsb.

9. A tough-elastic material based on starch as claimed in claim 1, characterized in that it comprises a present starch PS and a network-capable starch NS, wherein the proportion PNS of NS relative to NS and PS is 2-50 weight % dsb and wherein the network-capable starch NS is selected from SCA, LCA or a mixture of SCA and LCA and the present starch PS is selected from the group of oxidated starches, esterificated starches, etherificated starches, hydroxypropylated starches, hydroxyethylated starches, methyl starches, allyl starches, tripheylamethyl starches, carboxymethyl starches and crosslinked starches.

10. A tough-elastic material based on starch as claimed in claim 1, characterized in that it has a present starch PS and a network-capable starch NS, wherein the proportion PNS of NS relative to NS and PS is 2-50 in weight % dsb and wherein the network-capable starch NS is selected from SCA, LCA or a mixture of SCA and LCA and the present starch PS has a amylose content of less than 25%.

11. Soft capsule or soft capsule hull comprising the tough-elastic material based on starch as claimed in claim 1.

12. The soft capsule or soft capsule hull as claimed in claim 11, characterized in that the soft capsule is inserted and used similarly to conventional gelatine soft capsules and is manufactured using a continuous encapsulating process, wherein the capsule is formed similarly to gelatine encapsulation from films supplied symmetrically to the encapsulating plant, and these films are formed using current standard processes, such as an extrusion or casting process, wherein the encapsulating can take place directly from the freshly manufactured films or the films can be manufactured in advance.

13. The soft capsule or soft capsule hull as claimed in claim 12, characterized in that the continuous encapsulating process is a rotary-die process.

14. A hard capsule or hard capsule hull comprising the tough-elastic material based on starch, as claimed in claim 1.

15. Packaging based on the tough-elastic material based on starch, as claimed in claim 1.

16. Packaging and barriers for volatile materials such as perfumes and aromas, based on the tough-elastic material based on starch, as claimed in claim 1.

17. A molded article based on the tough-elastic material based on starch, as claimed in claim 1.

18. Use of the tough-elastic material based on starch, as claimed in claim 1, in the foodstuffs industry, galenics, cosmetics, health care, packaging or agricultural areas.
